(19) United States
(12) Patent Application Publication
(10) Pub. No.: US 2023/0321119 A1
(43) Pub. Date: Oct. 12, 2023

(54) STICK COMPOSITION CONTAINING CANNABINOIDS

(71) Applicant: Orcosa Inc., Ewing, NJ (US)

(72) Inventors: Simon WINSLOW, Bethlehem, PA (US); Vincent T MILETO, Flemington, NJ (US)

(73) Assignee: Orcosa Inc., Ewing, NJ (US)

(21) Appl. No.: 18/297,364

(22) Filed: Apr. 7, 2023

Related U.S. Application Data
(60) Provisional application No. 63/328,507, filed on Apr. 7, 2022.

(51) Int. Cl. A61K 31/00; A61K 9/06; A61K 47/44; A61K 31/355; A61K 9/00

(52) U.S. Cl. CPC A61K 31/658; A61K 9/06; A61K 47/44; A61K 31/355; A61K 9/0014

(57) ABSTRACT

A stick composition made of cannabidiol and/or tetrahydrocannabinol and waxes or solid-oils. The stick composition provides an effective dosage level of cannabidiol and/or tetrahydrocannabinol without unpleasant odors. The stick composition is applied topically for treating or reducing pain. In some instances, the cannabidiol and/or tetrahydrocannabinol may be the only active therapeutic agent present. The stick composition may also include one or more additives such as a vitamin, an essential oil, a flavorant, or a perfume.

### CROSS REFERENCE TO RELATED APPLICATIONS

This application claims the benefit of priority from U.S. Provisional Application No. 63/328,507 filed Apr. 7, 2022, the contents of which are incorporated herein by reference in their entirety.

### FIELD OF THE INVENTION

The present invention relates to stick compositions comprising cannabidiol, tetrahydrocannabinol or a combination of the same, and to methods of making and using such compositions.

### DISCUSSION OF THE BACKGROUND

There are many products currently available on the market that contain some level of cannabidiol (CBD), tetrahydrocannabinol (THC) or a combination of the two that purport to provide it in the form of a topical dosage for the treatment of pain. However, all these products suffer from one or more of the following drawbacks: 1) the claims of pain relief are unproven; 2) the claims of pain relief are unprovable due to the lack of manufacturing control; 3) the effective dosage levels of CBD and/or THC are relatively low; and 4) the odor from the CBD and/or THC make the product unpleasant to use.

There remains a need for improved cannabinoid-containing stick products which are effective at treating pain, which can be reliably and reproducibly manufactured, and/or which are not unpleasant to use.

Accordingly, one aspect of the present invention is a stick composition comprising cannabidiol, which is effective at treating pain, which can be reliably and reproducibly manufactured, and/or which is not unpleasant to use.

### SUMMARY OF THE INVENTION

The present invention relates to stick compositions comprising (1) cannabidiol and/or tetrahydrocannabinol and (2) waxes and/or solid-oils. Preferably, stick compositions disclosed herein have one or more of the following: the stick compositions are “anhydrous,” “devoid of water” or “free of water”; the stick compositions are “devoid of fluid oils” or “free of fluid oils” (not including antioxidants, fragrances, flavorants, essential oils); the stick compositions consist of, or consist essentially of, waxes or solid oils (not including antioxidants, fragrances, flavorants, essential oils.

The present invention also relates to methods of treating or reducing pain in a body location experiencing pain by topically applying compositions of the present invention to the body location to provide sufficient cannabidiol and/or tetrahydrocannabinol to the body location to treat or reduce pain.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8% -2.2%).

“A” or “an” as used herein means “at least one.”

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Wax” or “solid oil” as used herein is a lipophilic fatty compound that is solid at ambient temperature (25° C.) and at atmospheric pressure (760 mmHg), and changes from the solid to the liquid state reversibly, having a melting temperature of more than 25° C. and, for example, more than 30° C., and more than 40° C.

“Oil” or “non-solid oil” or “fluid oil” as used herein is a lipophilic fatty compound that is not solid at ambient temperature (25° C.) and at atmospheric pressure (760 mmHg).

“Solid” as used herein means the state of the composition at ambient temperature (25° C.) and at atmospheric pressure (760 mmHg), which conserves its form during storage. In contrast to “fluid” compositions, it does not flow under its own weight. It is advantageously characterized by a hardness (for example, it has a hardness of more than 0.5 MPa at ambient temperature and at atmospheric pressure).

“Free” or “devoid” of as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of water” means that water is preferably omitted (that is 0% by weight), and “devoid of water” means that less than about 1% water by weight based on the total weight of the composition is present. “Anhydrous” similarly means that little to no water is in the composition in accordance with general understanding of this term in the art.

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The compositions and methods of the present invention can comprise, consist of, or consist essentially of the essential elements and limitations of the invention described herein, as well as any additional or optional ingredients, components, or limitations described herein or otherwise useful.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

Preferably, stick compositions disclosed herein have one or more of the following:

the stick compositions are “anhydrous,” “devoid of water” or “free of water”;

the stick compositions are “devoid of liquid oils” or “free of liquid oils” (not including antioxidants, fragrances, flavorants, essential oils discussed above);

the stick compositions consist of, or consist essentially of, waxes or solid oils (not including antioxidants, fragrances, flavorants, essential oils discussed above); and/or

the stick compositions comprise cannabidiol and/or tetrahydrocannabinol.

### Cannabidiol

According to the present invention, stick compositions comprising cannabidiol oil are provided.

Preferably, the stick composition is formulated with, as the active therapeutic ingredient, cannabidiol (CBD), or any pharmaceutically acceptable derivative/analog, salt, solvate, or stereoisomer thereof. In some preferred embodiments, CBD or a derivative/analog thereof is the only active therapeutic ingredient in the stick composition. In some preferred embodiments, CBD is the only active therapeutic ingredient in the stick composition. In some preferred embodiments, a CBD derivative/analog is the only active therapeutic ingredient in the stick composition. In other embodiments, CBD or derivative/analog thereof may be combined with other active therapeutic ingredients such as THC or other pain relief active ingredients.

Preferably, stick compositions are formulated with CBD having a purity of at least 85 wt. %, preferably at least 86 wt. %, preferably at least 87 wt. %, preferably at least 88 wt. %, preferably at least 89 wt. %.

Preferably stick compositions are formulated with a solid form of CBD having a purity up to 91 wt. %, preferably up to 92 wt. %, preferably up to 93 wt. %, preferably up to 94 wt. %, preferably up to 95 wt. %, preferably up to 96 wt. %, preferably up to 97 wt. %, preferably up to 98 wt. %, and in the example provided below approximately 90 wt. %. The percent purity of CBD refers to the percent of CBD by mass relative to a total weight of CBD containing material—the CBD containing material being the sum of CBD plus any additional impurities which may be present, such as those impurities originating from the biomass from which the CBD is obtained (e.g., Cannabis sativa L./“Industrial Hemp”) or encountered during manufacture. The purity may be determined by methods known to those of ordinary skill in the art, for example, one or more of liquid chromatography such as high performance liquid chromatography (HPLC), liquid chromatography-mass spectrometry (LCMS), and liquid chromatography with tandem mass spectrometry (LCMSMS); gas chromatography such as headspace gas chromatography with flame ionization detection (HS-GC-FID), gas chromatography mass spectrometry (GC/MS), and headspace gas chromatography-mass spectrometry (HSGCMS); inductively coupled plasma-mass spectrometry (ICP-MS); and polymerase chain reaction (PCR).

Examples of potential impurities, such as those originating from the biomass from which the CBD is obtained (e.g., Cannabis sativa L./“Industrial Hemp”) or encountered during manufacture, include, but are not limited to,

cannabinoids (other than CBD) including, but not limited to, cannabidivarin (CBDV), cannabichromene (CBC), cannabidiolic acid (CBDa), cannabigerol (CBG), cannabigerolic acid (CBGa), cannabinol (CBN), tetrahydrocannabinolic acid (THCa), tetrahydrocannabivarin (THCV), tetrahydrocannabivarin acid (THCVa), and tetrahydrocannabinol (Δ9-THC) and related THC-cannabinoids such as Δ8-THC;

pesticides including, but not limited to, aldicarb, carbofuran, chlordane, chlorfenapyr, chlorpyrifos, coumaphos, daminozide, dichlorvos (DDVP), dimethoate, ethoprophos, etofenprox, fenoxycarb, fipronil, imazalil, methiocarb, methyl parathion, paclobutrazol, propoxur, spiroxamine, and thiacloprid;

residual solvents including, but not limited to, 1,4-dioxane, 2-butanol, 2-ethoxyethanol, 1,2-dichloroethane, acetone, acetonitrile, benzene, butane, cumene, cyclohexane, chloroform, ethanol, ethyl acetate, ethyl benzene, ethylene oxide, ethylene glycol, ethyl ether, heptane, isopropanol, methanol, methylene chloride, hexanes, isopropyl acetate, pentanes, propane, toluene, tetrahydrofuran, trichloroethene, and xylenes;

microbials including, but not limited to, Aspergillus flavus, Aspergillus fumigatus, Aspergillus niger, Aspergillus terreus, Salmonella, and Shiga toxin-producing E. coli;

mycotoxins including, but not limited to, aflatoxins (e.g., aflatoxin B1, aflatoxin B2, aflatoxin G1, and aflatoxin G2) and ochratoxin A;

heavy metals including, but not limited to, arsenic, cadmium, lead, and mercury;

terpenes including, but not limited to, (1) monoterpenes such as camphene, camphor, 3-carene, a-cedrene, cedrol, endo-fenchyl alcohol, eucalyptol, fenchone, geraniol, geranul acetate, hexahydrothymol, isoborneol, isopulegol, limonene, linalool, p-mentha-1,5-diene, β-myrcene, α- and β-pinene, pulegone, sabinene and hydrate, α- and γ-terpinene, terpineol, terpinolene, α-, β-, and γ-terpineol, nerol, borneol, and ocimene isomers I and II, and (2) sesquiterpenes such as α-bisabolol, β-caryophyllene, caryophyllene oxide, guaiol, α-humulene, cis- and trans-nerolidol, and valencene;

and mixtures thereof.

In some embodiments, the stick composition is formulated with a form of CBD which contains less than 1 wt. %, preferably less than 0.5 wt. %, preferably less than 0.1 wt. %, preferably less than 0.05 wt. %, preferably less than 0.001 wt. %, preferably 0 wt. % of the above listed impurities, based on a total weight of the CBD material, with specific mention being made to THC. In some embodiments, the stick composition is formulated with a form of CBD which contains no impurity, such as those listed above, in an amount above the limits of detection (LOD) and/or limits of quantification (LOQ) for the technique/instrumentation being used to make such a determination. For example, preferred stick compositions are those formulated with a pure form of CBD which has a THC content of less than 0.1577 wt. %, preferably less than 0.1 wt. %, preferably less than 0.01 wt. %, preferably less than 0.001 wt. %, based on a total weight of the CBD material. In preferred embodiments, the stick composition is formulated with a pure form of CBD which consists of, or consists essentially of, CBD.

Any CBD manufacturing method known by those of ordinary skill in the art which provides CBD of sufficient purity, may be utilized herein for preparation of the preferred CBD ATI. For illustration purposes, one exemplary CBD manufacturing method is described below, although it should be understood that numerous modifications and variations are possible, and the CBD may be produced using methods or techniques otherwise than as specifically described.

CBD may be extracted/isolated from biomass, for example, a cured flower of Cannabis sativa L. The biomass may contain, for example, at least 1 mg/g, preferably at least 2 mg/g, preferably at least 3 mg/g, and up to 10 mg/g, preferably up to 8 mg/g, preferably up to 6 mg/g, preferably up to 4 mg/g of CBD; at least 50 mg/g, preferably at least 60 mg/g, preferably at least 70 mg/g, preferably at least 80 mg/g, preferably at least 90 mg/g, and up to 150 mg/g, preferably up to 140 mg/g, preferably up to 130 mg/g, preferably up to 120 mg/g, preferably up to 110 mg/g, preferably up to 100 mg/g of cannabidiolic acid (CBDa); and no detectable amount of THC. Extraction of the biomass with an alcoholic solvent (e.g., ethanol) and cooling may form a tincture. The tincture may be filtered to remove sediment and particulates, and concentrated, for example, using a rotary evaporator.

An aluminum phyllosilicate clay (e.g., bentonite) may then be mixed with the concentrated product at a weight ratio of at least 2:1, preferably at least 3:1, preferably at least 4:1, and up to 6:1, preferably up to 5:1, and the resulting mix filtered to remove fats, waxes, and lipids. The product may then be frozen/winterized, after which the frozen product may be again filtered and taken through another solvent removal/recovery cycle to form a winterized crude.

Decarboxylation of the winterized crude by heating, for example in an induction oven centrifugal reactor, may be performed to remove the carboxylic acid functionality from the cannabinoids. Distillation of the decarboxylated material may then provide a distillate.

Also contemplated for use as an active therapeutic ingredient are derivatives/analogs of CBD that retain the desired activity for the treatment of pain. Derivatives/analogs that retain substantially the same activity as CBD, or more preferably exhibit improved activity, may be produced according to standard principles of medicinal chemistry, which are well known in the art. Such derivatives/analogs may exhibit a lesser degree of activity than CBD, so long as they retain sufficient activity to be therapeutically effective. Derivatives/analogs may exhibit improvements in other properties that are desirable in active therapeutic agents such as, for example, improved solubility, reduced toxicity, enhanced uptake, increased bioavailability, etc. Contemplated CBD derivatives/analogs include, but are not limited to, cannabidiolic acid compounds and variants thereof, such as cannabidiolic acid and esters of cannabidiolic acid, in particular alkyl esters of cannabidiolic acid (e.g., cannabidiolic acid methyl ester); 5′ side chain modified CBD compounds such as cannabidivarin (CBDV), cannabidiol-dimethylheptyl (CBD-DMH), and 1,2-cannabidiol-dimethylheptyl (1,2-CBD-DMH); 7-methyl modified CBD compounds such as 7-carboxy cannabidiol (7-COOH-CBD) and 7-hydroxy cannabidiol (7-OH-CBD); hydrogenated CBD compounds such as 8,9-dihydrocannabidiol (H₂-CBD) and tetrahydrocannabidiol (H₄-CBD); halogenated CBD compounds such as 3′-chloro-CBD, 3′,5′-dichloro-CBD, 3′-bromo-CBD, 3′,5′-dibromo-CBD, 3′-iodo-CBD, and 3′,5′-diiodo-CBD; hydroxyl group modified CBD compounds such as desoxy-CBD and dimethylether CBD; cannabielsoin (CBE); machaeridiols A, B, and C; as well as any pharmaceutically acceptable salts, solvates, and/or stereoisomers of such compounds. When a CBD derivative/analog is used as the ATI in the disclosed stick composition, particular preference is given to cannabidiolic acid methyl ester.

It is contemplated that CBD or derivatives/analogs of CBD may be useful in combination. It is also contemplated that CBD or derivatives/analogs of CBD may be useful in combination with current Standards of Care for the treatment of pain as well as any that evolve over the foreseeable future. Specific dosages and dosing regimens would be based on physicians' evolving knowledge and the general skill in the art.

Preferably, through the use of 90% pure CBD oil, a final product with an overall CBD concentration of 6% can be achieved. This can result in a 2 oz stick having approximately 3360 mg of CBD, which is an order of magnitude higher than typical products, without noticeable unpleasant odor. Furthermore, using the techniques described herein, a final product concentration of 10% CBD, or higher, can be achieved, in the example described below, resulting in 5,660 mg of CBD in a similar 2 oz stick at 10% final concentration levels. Indeed, these techniques can achieve final product concentrations of as high as 20% CBD by weight.

### Tetrahydrocannabinol

As described above, a stick composition can be formulated using CBD as the only active therapeutic ingredient. However, similar compositions can also be formed using the techniques described herein using THC or any pharmaceutically acceptable derivative/analog, salt, solvate, or stereoisomer thereof and/or a combination of THC and CBD as the active therapeutic ingredients.

Preferably, stick compositions using THC as an active therapeutic ingredient are formulated with THC having a purity of at least 85 wt. %, preferably at least 86 wt. %, preferably at least 87 wt. %, preferably at least 88 wt. %, preferably at least 89 wt. %. Preferably stick compositions are formulated with a solid form of THC having a purity up to 91 wt. %, preferably up to 92 wt. %, preferably up to 93 wt. %, preferably up to 94 wt. %, preferably up to 95 wt. %, preferably up to 96 wt. %, preferably up to 97 wt. %, preferably up to 98 wt. %, and in the example provided below approximately 90 wt. %. The percent purity of THC refers to the percent of THC by mass relative to a total weight of THC containing material—the THC containing material being the sum of THC plus any additional impurities which may be present, such as those impurities originating from the biomass from which the THC is obtained or encountered during manufacture. The purity may be determined by methods known to those of ordinary skill in the art, for example, one or more of liquid chromatography such as high performance liquid chromatography (HPLC), liquid chromatography-mass spectrometry (LCMS), and liquid chromatography with tandem mass spectrometry (LCMSMS); gas chromatography such as headspace gas chromatography with flame ionization detection (HS-GC-FID), gas chromatography mass spectrometry (GC/MS), and headspace gas chromatography-mass spectrometry (HSGCMS); inductively coupled plasma-mass spectrometry (ICP-MS); and polymerase chain reaction (PCR).

Also contemplated for use as an active therapeutic ingredient are derivatives/analogs of THC that retain at least some of the pharmacological activity of THC, for example, through activation of cannabinoid receptors. Derivatives/analogs that retain substantially the same activity as THC, or more preferably exhibit improved activity, may be produced according to standard principles of medicinal chemistry, which are well known in the art. Such derivatives/analogs may exhibit a lesser degree of activity than THC, so long as they retain sufficient activity to be therapeutically or pharmacologically effective. Derivatives/analogs may exhibit improvements in other properties that are desirable in active therapeutic agents such as, for example, improved solubility, reduced toxicity, enhanced uptake, increased bioavailability, etc. Contemplated THC derivatives/analogs include, but are not limited to, isomeric derivatives/analogs of THC (e.g., (+)-trans-Δ⁹-tetrahydrocannabinol, (−)-trans-Δ⁸-tetrahydrocannabinol); hydroxy derivatives/analogs (e.g., 11-hydroxy-Δ⁹-tetrahydrocannabinol, 11-hydroxy-Δ⁸-tetrahydrocannabinol, ΔM-919, AM-926, AM-938, AM-2389, 11-nor-9β-hydroxyhexahydrocannabinol, 11-nor-9β-hydroxyhexahydrocannabinol-DMH, HU-210, HU-211, desacetyl-L-nantradol, JWH-051); oxo derivatives/analogs (e.g., nabilone); carboxy derivatives/analogs (e.g., 11-nor-9-carboxy-THC, ajulemic acid); alkyl side chain modified derivatives/analogs (e.g., (−)-trans-Δ⁹-tetrahydrocannabinol-DMH, (−)-trans-Δ⁸-tetrahydrocannabinol-DMH, dimethylheptylpyran, tetrahydrocannabivarin, 5′-azido-Δ⁸-tetrahydrocannabinol, AMG-1, AMG-3, AM-41I, AM-087, L-759,633, O-1184); conformationally restricted side chain derivatives/analogs (e.g., AM-855); side-chain ester derivatives/analogs (e.g., AM7438); cannabinol (CBN); CP cannabinoids (e.g., CP 47,497, CP 55,940, CP 55,244, CP 50,556-1); prodrugs of THC such as ester, oxygenated ester, oxaester, pegylated ester, hydroxylated ester, branched hydroxylated ester, succinic acid monoester, oxalic acid mixed pegylated ester, amino ester, cyclic amino ester, acylated amino ester, carbonate, oxygenated carbonate, oxacarbonate, pegylated carbonate, hydroxylated carbonate, branched hydroxylated carbonate, aminoalkyl carbonate, cyclic aminoalkyl carbonate, acylated aminoalkyl carbonate, hydroxycarbonylalkyl carbonate, carbamate, alkyl carbamate, aminoalkyl carbamate, acylated aminoalkyl carbamate, cyclic aminoalkyl carbamate, oxacarbamate, pegylated carbamate, hydroxylated carbamate, branched hydroxylated carbamate, hydroxycarbonylalkyl carbamate, dihydrogen phosphate, alkali metal phosphate salt, alkaline earth metal phosphate salt, and phosphate salt of organic base prodrugs of THC, e.g., those disclosed in U.S. Pat. No. 9,957,246—incorporated herein by reference in its entirety; as well as any pharmaceutically acceptable salts, solvates, and/or stereoisomers of such compounds.

In certain embodiments the weight percentage of CBD, THC, and/or the combination of CBD and THC in the final product is preferably at least 5%, preferably at least 7%, preferably at least 8%, preferably at least 9%, preferably at least 10%, preferably at least 11%, preferably at least 12%, preferably at least 13%, preferably at least 15%, and preferably up to 10%, preferably up to 13%, preferably up to 14%, preferably up to 15%, preferably up to 16%, preferably up to 17%, and preferably up to 20%. For example, this would include ranges of 5% to 10%, 5% to 15%, 5% to 20%, 10% to 15%, 10% to 20%, and 15% to 20% by weight with respect to the total weight of the composition. These weight percentages relate to compositions containing CBD alone, THC alone, or a combination of THC and CBD.

In compositions of the present invention containing both THC and CBD, highly pure CBD oil and highly pure THC oil have similar properties and behavior, such that mixtures of the two active ingredients can be made according to the present teachings by substituting high-purity THC oil for high-purity CBD oil and vice versa. For example, in Example 1 described below a stick formulation with 10% CBD oil by weight is described. By way of example, the same composition could be made with 5% by weight CBD oil and 5% by weight THC oil, with other ingredients remaining constant. Or a different mixture of CBD oil and THC oil could be used with the combined mixture accounting for 10% by weight of the final product. For instance, a mixture of CBD oil and THC oil may provide an amount of CBD that is at least 10%, at least 20 wt %, at least 30 wt %, at least 40 wt %, at least 50 wt %, at least 60 wt %, at least 70 wt %, at least 80 wt %, and/or at most 90 wt %, at most 80 wt %, at most 70 wt %, at most 60 wt %, at most 50 wt %, at most 40 wt %, at most 30 wt %, at most 20 wt %, relative to a combined mass of the CBD and THC in the composition. Similarly, a mixture of CBD oil and THC oil may provide an amount of THC that is at least 10%, at least 20 wt %, at least 30 wt %, at least 40 wt %, at least 50 wt %, at least 60 wt %, at least 70 wt %, at least 80 wt %, and/or at most 90 wt %, at most 80 wt %, at most 70 wt %, at most 60 wt %, at most 50 wt %, at most 40 wt %, at most 30 wt %, at most 20 wt %, relative to a combined mass of the CBD and THC in the composition.

### Waxes/Solid Oils

According to present invention, compositions comprising waxes and/or solid oils are provided. Each or all of the waxes and/or solid oils can be hydrocarbon, fluorinated and/or silicone, and be of plant, mineral, animal and/or synthetic origin.

Suitable examples of solid oils that can be used in accordance with the present disclosure include natural solid oils, although the solid oils are not required to be natural. Such natural oil(s) are oils recovered or extracted from foods, and/or preferably oils recovered from plants or other vegetative life (as opposed to oils which are derived from natural sources through reactions and which are not “natural oil(s)” as used herein). For example, plant oils include glyceride triesters, which are generally triesters of fatty acids and of glycerol, the fatty acids of which can have varied chain lengths from C4 to C24, it being possible for these chains to be saturated or unsaturated and linear or branched. Non-limiting examples of suitable natural solid oil(s) include shea butter, cocoa butter, coconut oil, and palm oil.

Suitable examples of waxes that can be used in accordance with the present disclosure include those generally used in the cosmetics field: they include those of natural origin, such as beeswax, carnauba wax, candelilla wax, ouricoury wax, Japan wax, cork fibre wax or sugar cane wax, rice wax, montan wax, paraffin wax, lignite wax or microcrystalline wax, ceresin or ozokerite, and hydrogenated oils such as hydrogenated castor oil or jojoba oil; synthetic waxes such as the polyethylene waxes obtained from the polymerization or copolymerization of ethylene, and Fischer-Tropsch waxes, or else esters of fatty acids, such as octacosanyl stearate, glycerides which are concrete at 30° C., for example at 45° C., silicone waxes, such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, or else di(1,1,1-trimethylolpropane) tetrastearate, which is sold or manufactured by Heterene under the name HEST 2T-4S, and mixtures thereof.

Further suitable examples of wax include, but are not limited to, BIS-PEG-12 DIMETHICONE CANDELILLATE wax such as for example the Siliconyl Candelilla Wax marketed by the company KOSTER KEUNEN, hydrogenated Jojoba wax such as for example that marketed by the company DESERT WHALE, hydrogenated palm oil such as that marketed by the company SIO, rice bran wax, Sumac wax, ceresin waxes, laurel wax, Chinese insect wax, Shellac wax, hydrogenated olive oil such as Waxolive from the company SOLIANCE, waxes obtained by hydrogenation of olive oil esterified with C12 to C18 chain fatty alcohols such as those sold by the company SOPHIM under the brand names Phytowax Olive 12L44, 14L48, 16L55 and 18L57, waxes obtained by hydrogenation of castor oil esterified with cetyl or behenyl alcohol such as for example those which are sold under the names Phytowax Ricin 16 L 64 and Phytowax Ricin 22 L 73 by the company SOPHIM, hydrogenated Cameline wax, Ouricury wax, Montan wax, ozokerite waxes such as for example Wax SP 1020 P marketed by the company Strahl & Pitsch, microcrystalline waxes such as for example that sold under the brand name Microwax HW by the company PARAMELT, triglycerides of lauric, palmitic, cetylic and stearic acids (INCI name: hydrogenated coco glycerides) such as for example that sold under the brand name Softisan 100 by the company SASOL, polymethylene waxes such as for example that sold under the brand name Cirebelle 303 by the company SASOL, polyethylene waxes such as for example those sold under the brand names Performalene 400 polyethylene, Performalene 655 polyethylene and Performalene 500-L polyethylene by the company New Phase Technologies, alcohol-polyethylene waxes such as for example that marketed under the name Performacol 425 Alcohol by the company BARECO, the 95/5 ethylene/acrylic acid copolymer sold under the brand name AC 540 wax by the company Honeywell, hydroxyoctacosanyl hydroxy-stearate such as for example that sold under the brand name Elfacos C 26 by the company AKZO, octacosanyl stearate such as for example that marketed under the name Kester Wax K 82H by the company KOSTER KEUNEN, stearyl stearate such as for example that marketed under the name Liponate SS by the company LIPO CHEMICALS, pentaerythritol distearate such as for example that marketed under the name Cutina PES by the company COGNIS, the mixture of dibehenyl adipate, dioctadecyl adipate and di-eicosanyl adipate (INCI name C18-C22 dialkyl adipate), the mixture of dilauryl adipate and ditetradecyl adipate (INCI name: C12-C14 dialkyl adipate), the mixture of dioctadecyl sebacate, didocosyl sebacate and dieicosyl sebacate (INCI name: C18-C22 dialkyl sebacate) and the mixture of dioctadecyl octadecanedioate, didocosyl octanedioate and dieicosyl octanedioate (INCI name: C18-C22 dialkyl octanedioate) such as for example those marketed by the company COGNIS, pentaerythrityl tetrastearate such as for example Liponate PS-4 from the company Lipo Chemicals, tetracontanyl stearate such as for example Kester Wax K76H from the company KOSTER KEUNEN, stearyl benzoate such as for example Finsolv 116 from the company FINETEX, behenyl fumarate such as for example Marrix 222 from the company AKZO BERNEL, di-(trimethylol-1,1,1-propane) tetrastearate such as for example that which is offered under the name “HEST 2T-4S” by the company HETERENE, didotriacontanyl distearate such as for example Kester Wax K82D from the company KOSTER KEUNEN, polyethylene glycol montanate with 4 ethylene oxide units (PEG-4) such as for example that which is sold under the brand name Clariant Licowax KST1, hexanediol disalicylate such as for example Betawax RX-13750 marketed by the company CP Hall, dipentaerythritol hexastearate such as for example that which is sold under the brand name Hest 2P-6S by the company HETERENE, ditrimethylolpropane tetrabehenate such as for example that which is sold under the brand name Hest 2T-4B by the company HETERENE, Jojoba esters such as for example that which is sold under the brand name Floraester HIP by the company FLORATECH, mixtures of linear (C20-40) carboxylic acid/saturated hydrocarbons (INCI name: C20-40 acid polyethylene) such as for example Performacid 350 acid from the company NEW PHASE TECHNOLOGIES, synthetic wax of the Fischer-Tropsch type such as that marketed under the name Rosswax 100 by the company ROSS, cetyl alcohol, stearyl alcohol, behenyl alcohol, dioctadecyl carbonate such as for example Cutina KE 3737, saccharose polybehenate such as for example Crodaderm B from the company CRODA, waxes of plant origin such as carnauba wax, candelilla wax, hydrogenated jojoba wax, sumac wax, waxes obtained by hydrogenation of olive oil esterified with C12 to C18 chain fatty alcohols sold by the company SOPHIM in the Phytowax range (12L44, 14L48, 16L55 and 18L57), rice bran wax, cetyl, stearyl and behenyl alcohols, laurel wax, Ouricury wax and mixtures thereof can be mentioned.

Preferably, the waxes and/or solid oils constitute the base of the stick composition and comprise the vast majority of the stick composition—preferably, the waxes and/or solid oils are present in a total amount ranging from about 50% to about 97.55% by weight, preferably from about 70% to about 96% by weight, and preferably from about 80% to about 95% by weight, based on the total weight of the composition, including all ranges and subranges within these ranges.

### Additional Additives

The composition of the invention can also comprise any additive usually used in the field under consideration. For example, dispersants such as antioxidants, film forming agents, oils, essential oils, sunscreens, preserving agents, fragrances, flavorants, fillers, neutralizing agents, cosmetic and dermatological active agents such as, for example, emollients, moisturizers, vitamins, essential fatty acids, surfactants, and mixtures thereof can be added, by way of example.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency, texture or to enhance CBD transport into and/or across the dermal layer of skin. For example, in the example below, vitamin E is added to the composition which enhances transfer of the CBD into and/or across the dermal layer after application to skin.

These additives may be present in the composition in a proportion from 0% to 10% (such as from 2% to 8%) relative to the total weight of the composition and further such as from 0.1% to 1% (if present), including all ranges and subranges therebetween. For instance, one or more additives may be present in the composition at a weight percent concentration of at least 0.001 wt %, at least 0.01 wt %, at least 0.1 wt %, at least 0.5 wt %, at least 1 wt %, at least 1.5 wt %, at least 2 wt %, at least 3 wt %, at least 4 wt %, at least 5 wt %, at least 6 wt %, at least 7 wt %, at least 8 wt %, at least 9 wt %, and/or at most 10 wt %, at most 9 wt %, at most 8 wt %, at most 7 wt %, at most 6 wt %, at most 5 wt %, at most 4 wt %, at most 3 wt %, at most 2 wt %, at most 1 wt %, at most 0.5 wt %, at most 0.1 wt %, at most 0.01 wt %, relative to a total weight of the composition.

Needless to say, the composition of the invention should be cosmetically or dermatologically acceptable, i.e., it should contain a non-toxic physiologically acceptable medium.

According to preferred embodiments of the present invention, methods of treating or reducing pain in a body location experiencing pain by topically applying compositions of the present invention to the body location to provide sufficient cannabidiol to the body location to treat or reduce pain are provided. In accordance with this embodiment, the compositions of the present invention are applied topically to the desired body area (preferably human) in a manner which provides pain treating or pain reducing effective amounts of cannabidiol to the area. The compositions may be applied to the desired area as needed, preferably once or twice daily, more preferably once daily and then preferably allowed to dry before subjecting the area to contact such as with an object such as clothing.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example I—Sample Formulations

| | | AMOUNT | PREFERRED |
|---|---|---|---|
| | | RANGE | AMOUNT |
| INGREDIENT | | (by weight of | (by weight of |
| CATEGORY | INGREDIENT | composition) | composition) |
| Waxes/Solid Oils | Coconut oil | 40-60% | About 50% |
| | Beeswax | 25-40% | About 33% |
| | CBD oil | 5-20% | About 10% |
| Antioxidant | Vitamin E | 1-10% | About 5% |
| Flavorant/Perfume | Vanilla Creme | 50-250 | 120-180 |
| | | drops(<1%) | drops(<1%) |
| Essential Oils | Lavender, Tea Tree | 50-250 | 120-180 |
| | | drops(<1%) | drops(<1%) |

### Example II—Composition Preparation

The composition in Example I can be prepared in the following manner:

Prepare a pot or vessel having an indirect heat source (e.g., water jacket or water bath) at a temperature above the melting point of all waxes/solid oils in the composition.

Sequentially add the waxes/solid oils to the pot or vessel with melting and stirring. For example, (1) add coconut oil to the pot, melt and stir the coconut oil; (2) Add beeswax to the pot, melt and stir the beeswax; (3) Add CBD oil to the pot, melt and stir the CBD oil (if necessary); and (4) Add any remaining solid ingredients such as antioxidants/essential oils/flavorants/perfume to the pot with melting and heating (if necessary).

After solids have been added to the pot and melted, add non-solid ingredients such as antioxidants/essential oils/flavorants/perfume to the pot with stirring (if necessary).

Mix until homogeneous; and

Cool the homogeneous mixture and form a solid stick composition.

1. A composition, comprising:
(a) cannabidiol in an amount of from 5% to 20% by weight of the total weight of the composition, and
(b) at least one wax and/or at least one solid oil in an amount of from 50% to 97.55% by weight of the total weight of the composition,
wherein the composition is in the form of a solid stick.

2. The composition of claim 1, wherein the cannabidiol is the only active therapeutic agent in the composition.

3. The composition of claim 1, wherein the cannabidiol has at least 80% purity.

4. The composition of claim 3, wherein the cannabidiol has at least 80% and up to 95% purity.

5. The composition of claim 3, wherein the cannabidiol has at least 80% and up to 92% purity.

6. The composition of claim 1, wherein the composition is devoid of water.

7. The composition of claim 1, wherein the composition is free of water.

8. The composition of claim 1, further comprising at least one essential oil.

9. The composition of claim 1, further comprising at least one flavorant and/or at least one perfume.

10. The composition of claim 1, further comprising at least one antioxidant.

11. The composition of claim 10, wherein the least one antioxidant is at least one vitamin.

12. The composition of claim 11, wherein the at least one vitamin is Vitamin E.

13. The composition of claim 12, wherein the Vitamin E is present in an amount of from 1% to 10% by weight of the total weight of the composition.

14. The composition of claim 13, wherein the Vitamin E is present in an amount of from 4% to 6% by weight of the total weight of the composition.

15. The composition of claim 1, wherein the at least one wax and/or at least one solid oil is present from 80% to 95% by weight with respect to the total weight of the composition.

16. A composition consisting of:
(a) cannabidiol in an amount of from 5% to 20% by weight of the total weight of the composition,
(b) at least one wax and/or at least one solid oil,
(c) at least one vitamin,
(d) optionally at least one essential oil, and
(e) optionally at least one flavorant and/or at least one perfume,
wherein the composition is in the form of a solid stick.

17. A method of reducing pain in a body location experiencing pain comprising topically applying the composition of claim 1 to the body location to provide sufficient cannabidiol to the body location to reduce the pain.

18. A composition, comprising:
(a) tetrahydrocannabinol, or a combination of tetrahydrocannabinol and cannabidiol, in an amount of from 5% to 20% by weight of the total weight of the composition, and
(b) at least one wax and/or at least one solid oil in an amount of from 50% to 97.55% by weight of the total weight of the composition,
wherein the composition is in the form of a solid stick.

19. The composition of claim 18, wherein both tetrahydrocannabinol and cannabidiol are present, and
wherein an amount of tetrahydrocannabinol is in a range of 30 wt % to 70 wt % relative to a combined mass of the tetrahydrocannabinol and cannabidiol.
